(19) United States
(12) Patent Application Publication
(10) Pub. No.: US 2015/0313635 A1
(43) Pub. Date: Nov. 5, 2015

(54) DOUBLE PORT ARTHROSCOPIC CANNULA

(71) Applicant: Amir A. Jamali, Berkeley, CA (US)

(72) Inventor: Amir A. Jamali, Berkeley, CA (US)

(21) Appl. No.: 14/704,910

(22) Filed: May 5, 2015

Related U.S. Application Data
(60) Provisional application No. 61/988,681, filed on May 5, 2014.

(51) Int. Cl. A61B 17/34; A61B 1/317; A61B 1/00

(52) U.S. Cl. CPC A61B 17/3423; A61B 1/00154; A61B 1/317; A61B 2017/3445; A61B 2017/00238

(57) ABSTRACT

A single shaft double port cannula having a shaft with discrete and separated channels to provide a method and apparatus that allows entry and passage of any two elongate surgical instruments through a single cannula.

### CROSS REFERENCES TO RELATED APPLICATIONS

The present application claims the benefit of the filing date of U.S. patent application Ser. No. 61/988,681, filed May 5, 2014 (May 5, 2014), incorporated in its entirety by reference herein.

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention relates generally to a cannula for use in arthroscopic surgery and endoscopic procedures, and more particularly to a novel double port cannula that facilitates arthroscopy of synovial joints and endoscopic procedures on the abdomen and spine.

2. Background Discussion

Arthroscopy has revolutionized the treatment of joint diseases. In the knee, arthroscopy has allowed for minimally invasive treatments of meniscal tears and cartilage pathology and facilitated other more extensive surgeries such as ACL reconstruction and meniscal transplantation. Arthroscopy has been expanded into other fields such as shoulder, hip elbow, ankle, and even the wrist. In joints, such as the hip and knee in particular, arthroscopic suturing techniques are particularly helpful for repair of the labrum and capsule. However, suture management can occasionally challenge even an experienced arthroscopist. The problem is one of access. In the hip, for instance, the presence of the femoral head and the depth of the joint can make it nearly impossible to access certain areas except with one cannula. Thus, it is impossible to implement a surgical action in that zone. In the spine, muscle-sparing approaches have been developed in the removal of disc material and the fusion of vertebral bones in the treatment of disc disease. However, access is limited to one to two pathways to the diseased area.

### SUMMARY OF THE INVENTION

The current invention has been developed to provide a method and apparatus to address the issue of access by providing two ports within a single double port cannula. The inventive double port mechanism allows entry and passage of any two elongate instruments, including an endoscope through one of the channels. This dramatically increases the options for achieving the surgical goals. The inventive double port cannula allows the surgeon to implement the surgical plan through the second port. The invention has applications to the shoulder for the repair of the rotator cuff where the sutures can be pulled through the second port and can help to apply traction of the cuff or store the sutures until they are ready to be tied. It can be utilized in the hip in the repair of the labrum or treatment of cartilage lesions. It can further be utilized in the knee for the repair of ligaments such as the anterior cruciate ligament or the menisci. The invention can be used in the spine for the treatment of intervertebral disc disease with a minimally invasive methodology with the endoscope entered through one channel or portal and the pathological material removed or repaired through the second portal.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 is a lower perspective end view of the inventive cannula;

FIG. 2 is a side view in elevation of the inventive cannula shown in FIG. 1;

FIG. 3 is an enlarged detailed cross-sectional view of the inventive cannula taken along section line 3-3 in FIG. 2;

FIG. 4 is an enlarged detailed view of the interior (distal) end portion of the inventive cannula shown in FIG. 1;

FIG. 5 is an upper perspective external (proximal) end view of the inventive cannula;

FIG. 6 is a rotated and enlarged view of the proximal end portion of the inventive cannula shown in FIG. 5;

FIG. 7A is an exploded lower perspective view of a second preferred embodiment of the inventive cannula, which includes a threaded exterior end portion and an insertion stylette (pin);

FIG. 7B is the same view showing the insertion pin inserted into the threaded cannula;

FIG. 8A is a front view in elevation of the insertion pin of the second preferred embodiment;

FIG. 8B is a side view in elevation thereof;

FIG. 8C is a top plan view thereof;

FIG. 8D is a bottom plan view thereof;

FIG. 9A is a side view in elevation of the cannula of the second preferred embodiment;

FIG. 9B is the same view with the cannula axially rotated 90 degrees;

FIG. 9C is a top plan view thereof;

FIG. 9D is a bottom plan view thereof;

FIG. 10A is a in elevation of the second preferred embodiment assembled showing the insertion pin front side; and

FIG. 10B is the same view with the insertion pin and cannula rotated axially 90 degrees.

### DETAILED DESCRIPTION OF THE INVENTION

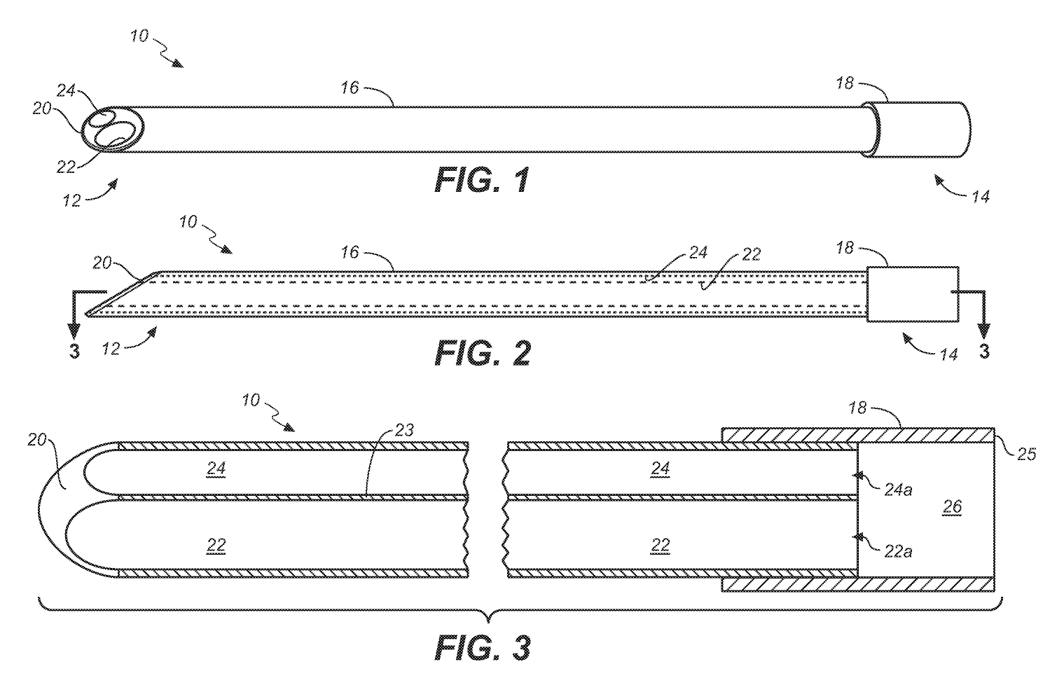

Referring first to FIGS. 1 through 6, wherein like reference numerals refer to like components in the various views, there is illustrated therein a first preferred embodiment of a new and improved cannula with two ports, generally denominated 10 herein.

As seen in FIGS. 1-3, in a first preferred embodiment the cannula of the present invention 10 preferably includes a cylindrical shaft 16, having a an interior end 12, truncated obliquely so as to form a beveled end or “point” 20, an exterior end 14, a collar or ferule 18 disposed on the exterior end, and first and second generally linear and parallel bores 22 and 24, separated by a partition 23 from the exterior end 14 to the interior end 12. The bores can be identical or variable in diameter.

FIG. 3 provides detail showing the shaft 16, an exterior end 14, the collar 18, and the interior end (or articular apparatus) 12 with the main channel opening 22 and the accessory channel opening 22. The collar 18 may include a rim 25 that extends beyond the openings of the channels and defines a cylindrical cup 26 to facilitate placement of an arthroscope or a larger instrument, typically inserted into the exterior end (outer) entry site into the main channel. Alternatively, smaller instruments (such as radiofrequency probes, suture passers or retracting sutures) can be passed through the accessory channel.

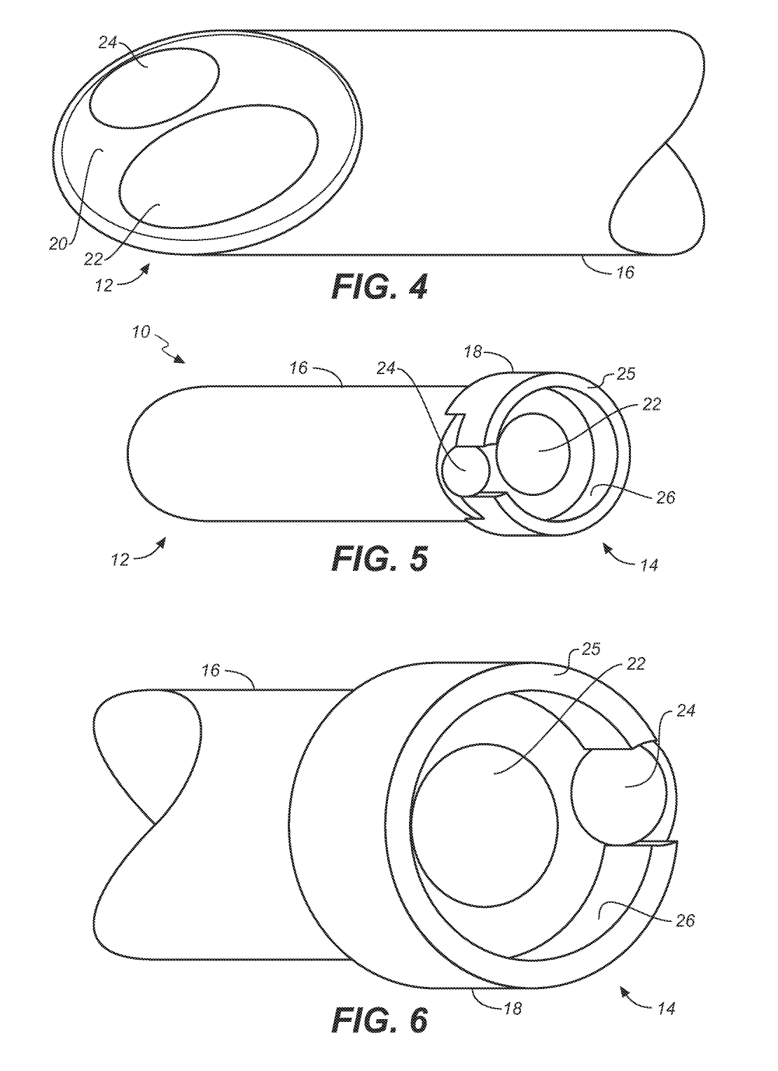

FIG. 4 provides detail concerning the double port cannula articular end tip 20. The beveled opening of the cannula 10 makes it able to accommodate a variety of endoscope/arthroscopes (the terms used interchangeably herein) through the interior end of the main channel 22 and other smaller instruments through the accessory channel 24 with a clear structural separation between the two channels of the cannula.

FIG. 5 is an end view in perspective showing the external end 14. In this view, the main cannula assembly is shown. Looking from outside the external end, thus outside the patient's body, there is an outer entry hole 22a for the main channel 22 and the accessory channel outer entry 24a for the accessory channel 24. An endoscope/arthroscope inserted into main channel 22 is maintained in position with a fluid seal using the endoscope/arthroscope attachment mechanism, collar 18.

FIG. 6 is a detailed end view of the double port cannula, again showing the exterior end 14, rotated 180 degrees from its position as seen in FIG. 5 so that accessory channel 24 is better appreciated.

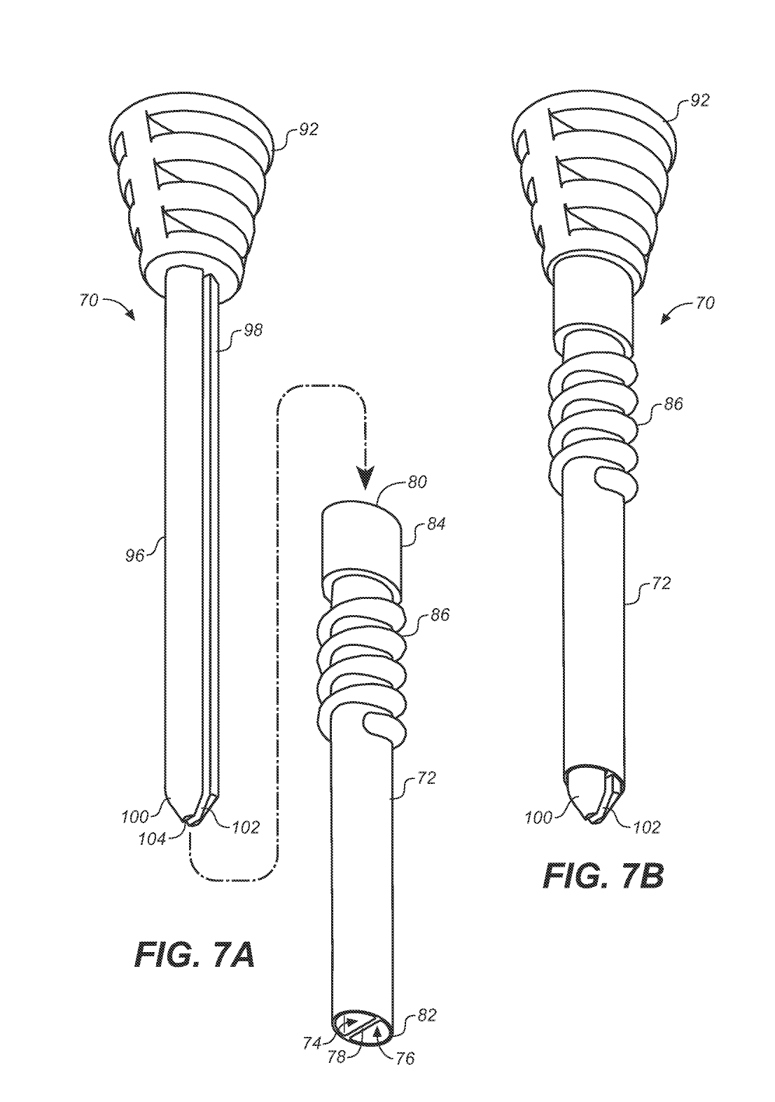

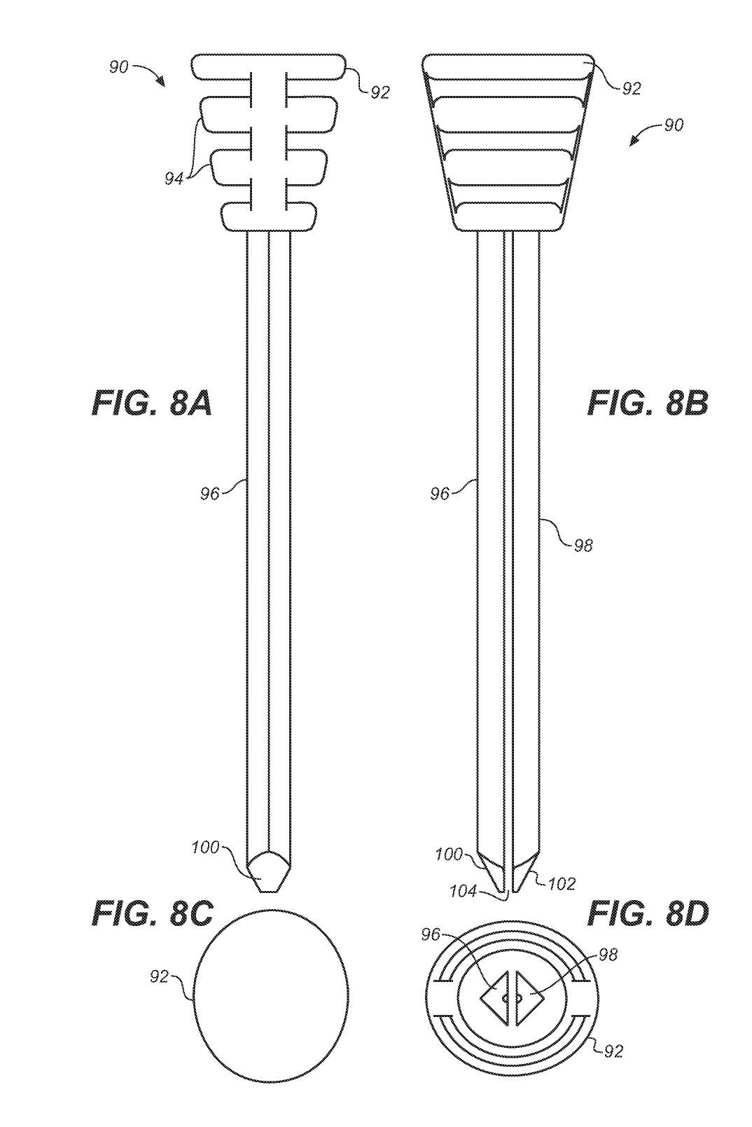

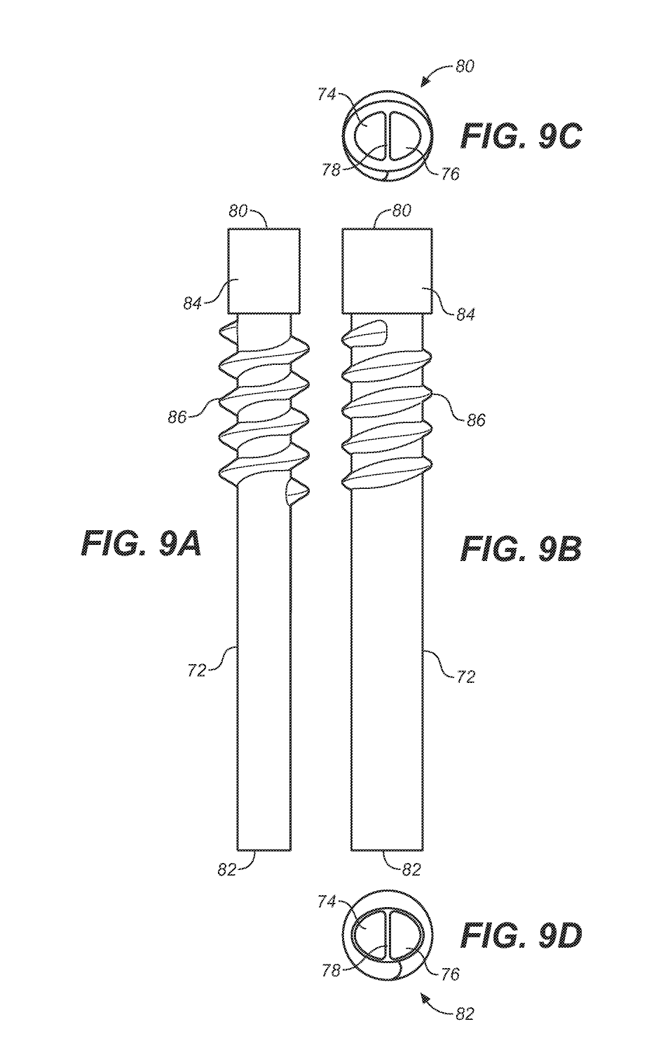

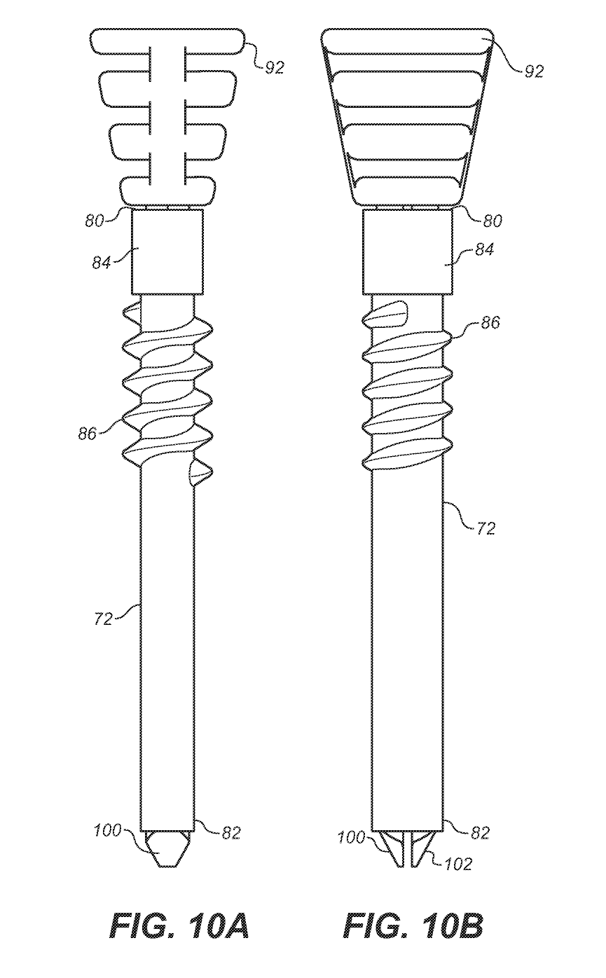

FIGS. 7A through 10B show a second preferred embodiment 70 of the inventive cannula. These views show, collectively, that in a second preferred embodiment, the cannula includes an elongate shaft or tube 72 having first and second through ports 74, 76, separated internally by a bifurcation or partition 78, and extending from an exterior (proximal) end 80 to an interior (distal) end 82. The exterior end 80 includes a collar or ferrule 84 that functions as an anthroscopic attachment structure. This embodiment further includes male threads 86, which maintain the cannula in position at the desired depth from the skin.

The second preferred embodiment next includes an insertion pin or stylette 90 having a head 92 with gripping elements 94, including, but not limited to, nubs, knurls, fins, ribs, or any suitable surface feature that facilitates a secure finger hold. Integrally connected to head 92 are first and second split prongs 96, 98, each configured for insertion into the through ports 74, 76 in cannula shaft 72 each extend to a beveled end 100, 102 which angle inwardly to form an effective point 104, which extends through the interior end 82 of cannula 72 when inserted. This facilitates insertion into a patient's body.

While the prongs are shown in the view having substantially the same dimensions, it will be appreciated that they may be sized for passage of different instruments while still achieving the functional features of the above-described insertion pin.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. An arthroscopic cannula, comprising a hollow shaft containing two separated and discrete channels to allow for passage of independent instruments through each channel.

2. The arthroscopic cannula of claim 1, wherein consisting of a length of between 10 cm and 300 cm

3. The arthroscopic cannula of claim 1, wherein each channel has a diameter of between 3 and 10 mm.

4. The arthroscopic cannula of claim 1, wherein said hollow shaft includes an interior end and an exterior end, and said exterior end includes an accessory attachment structure.

5. The arthroscopic cannula of claim 4, wherein said accessory attachment structure is a collar integrally disposed on said exterior end.

6. The arthroscopic cannula of claim 1, wherein said hollow shaft includes an interior end angled so as to form an effective point to facilitate insertion into a human joint or other body cavity or potential space.

7. The arthroscopic cannula of claim 1, wherein said hollow shaft includes an interior end and an exterior end, and said channels are separated by a partition extending from said interior end to said exterior end.

8. The arthroscopic cannula of claim 1, wherein said hollow shaft includes an interior end having a collar for securing an arthroscope/endoscope through one of said channels.

9. The arthroscopic cannula of claim 8, wherein said collar includes a rim that extends beyond the openings of said channels.

10. The arthroscopic cannula of claim 9, wherein said collar defines a cylindrical cup to facilitate placement of an arthroscope/endoscope or a larger instrument.

11. The arthroscopic cannula of claim 1, wherein said hollow shaft includes a beveled interior end.

12. An arthroscopic cannula, comprising a hollow shaft bifurcated by a partition into first and second ports extending from an exterior end to an interior end.

13. The cannula of claim 12, wherein said shaft includes a beveled interior end.

14. The cannula of claim 12, wherein said shaft includes accessory attachment structure on an exterior end.
